(19) United States
(12) Patent Application Publication
(10) Pub. No.: US 2009/0049611 A1
(43) Pub. Date: Feb. 26, 2009

(54) PATIENT-SECURING STRAP

(76) Inventors: Stefan Braunreuther, Kemnath (DE); Herbert Reisslein, Neunkirchen a.S. (DE)

Correspondence Address: BRINKS HOFER GILSON & LIONE, P.O. BOX 10395, CHICAGO, IL, 60610, US

(21) Appl. No.: 12/067,784

(22) PCT Filed: Aug. 3, 2006
(86) PCT No.: PCT/EP06/65044

§ 371 Date: Sep. 25, 2008

(30) Foreign Application Priority Data
Sep. 28, 2005 (DE) 10 2005 046 415.7

(51) Int. Cl. A47B 13/00; A61B 5/00

(52) U.S. Cl. 5/601; 600/301

(57) ABSTRACT

The invention relates to a patient-securing strap and to a patient-supporting device with a patient-securing strap. In one illustrative embodiment, the patient-securing strap is made mainly of plastic and has an electrical conductor made of substantially radioparent plastic, an electrical sensor which is connected to the conductor and which is made of a substantially radioparent material, and transmitter which is connected to the conductor and which is designed such that sensor signals output from the sensor to the conductor are sent to a data transmission line. In another illustrative embodiment, a patient-supporting device comprises one such patient-securing strap. The fact that the patient-securing strap is for the most part made of radioparent material means that it does not cause interfering image artefacts during an X-ray examination. It can therefore readily be used for an X-ray examination or CT scan of the patient and at the same time, by means of the electrical sensor, allows measurement values to be recorded. In other words, it is thus made possible to perform an X-ray examination while at the same time recording measurement values.

The present patent document is a nationalization of PCT Application Serial Number PCT/EP2006/065044, filed Aug. 3, 2006, designating the United States, which is hereby incorporated by reference. This application also claims the benefit of DE 10 2005 046 415.7, filed Sep. 28, 2005, which is hereby incorporated by reference.

### BACKGROUND

The present embodiments relate to a patient-securing strap.

Patient-supporting devices are used in medical diagnostics and therapies for transporting patients or performing particular diagnostic or therapeutic procedures (measures). For example, a patient-supporting device can be used for subjecting a patient to a CT, MR, or X-ray examination or for performing a radiation or ultrasound therapy. Depending on the patient's condition and type of procedure, the patient's body or parts thereof may be secured in position on the patient-supporting device, which may be referred to as a table. For instance, a patient's body may be secured in position on the patient-supporting device during a high-resolution MR examination or a surgical intervention, such as a biopsy in conjunction with an examination.

Patients acutely at risk (high risk patients) may include, for example, patients in an accident, emergency department, or intensive-care ward. Patients acutely at risk may require early diagnosis of a multiplicity of vital parameters and early application of imaging techniques, such as X-ray or CT imaging. For example, diagnosis of a multiplicity of vital parameters and imaging techniques may occur at the same time. Patients acutely at risk may first be examined for the major vital functions, such as pulse, heart rate, temperature, blood-oxygen saturation, blood pressure, and, where applicable, sweating or percentage of body fat. The vital functions may be confirmed (determined) in the ambulance if a patient is conveyed in one. Further measures, such as an X-ray examination, may not be initiated until the vital functions have undergone a preliminary examination. Time is lost because the individual examinations have to be conducted consecutively.

In medical practice, a specific patient table may be used for each examination process. The patient table for transporting the patient within an accident and emergency department differs from that used for examining using a CT device, which table differs from a patient table for an X-ray examination. Relocating a patient who is traumatized by an accident imposes a strain and is time-consuming. A patient table that may be used for transporting the patient and for a multiplicity of examination or therapeutic procedures (measures).

### SUMMARY

The present embodiments may obviate one or more of the drawbacks or limitations inherent in the related art. For example, in one embodiment, a patient-securing strap may be used to save time.

In one embodiment, a patient-securing strap may include an electric conductor, an electronic sensor, and a transmitter that is operable to transmit sensor signals fed out by the sensor onto the conductor to a data transmission link. The patient-securing strap may include (e.g., “be made substantially of”) plastic. The electric conductor may include (e.g., “be made substantially of”) X-ray-transparent plastic (material). The electric sensor may be linked to the conductor and include (e.g., “be made substantially of”) an X-ray-transparent material. The transmitter may be connected to the conductor.

In another embodiment, a patient-supporting device may include a patient-securing strap.

The patient-securing strap may be made for the most part of an X-ray-transparent material. The patient-securing strap may not give rise to any disruptive image artifacts during an X-ray examination. The patient-securing strap may readily be used for an X-ray or CT examination performed on the patient and allow measurements to be registered by the electric sensor. An X-ray examination may be conducted, and simultaneously (or subsequently) measurements may be registered. For example, the patient-securing strap does not need to be changed between the X-ray examination and registering measurements. The patient-securing strap may not produce any image artifacts when ultrasound, PET, or other diagnostic (or therapeutic) imaging techniques are used.

In one embodiment, the electric sensor measures a physiological parameter or vital function, for example, a pulse, a heart rate, a body temperature, an amount of body perspiration, a blood-oxygen saturation, a percentage of body fat, or a blood pressure. The physiological parameter or vital function may be used to diagnose one or more vital parameters to be carried out at the same time as an X-ray or CT examination. It will hence be possible to avoid performing the examinations consecutively, step-by-step and save time.

In one embodiment, the patient-securing strap includes a plurality of electric sensors. The plurality of electric sensors may measure, for example, at least all major vital functions whose diagnosis ought to be available immediately for acute measures. For example, sensors for pulse, heart rate, blood-oxygen saturation, and blood pressure could be provided simultaneously. A patient-securing strap may be applied in the ambulance and perform the pre-diagnosis, such that the patient may be subjected (e.g., immediately) to an X-ray or CT examination. The patient may be emplaced directly in a respective examination device and the vital functions measured when the patient is being or has been emplaced in the examination device.

In one embodiment, the data transmission link is cableless, and the transmitter is a transponder. While allowing signals to be conveyed within the scope of a transmitting functionality, a transponder transmits only in response to a transmission request that it receives within the scope of a receiving functionality. The transponder may operate without a power supply, so that using a transponder will enable the patient-securing strap to be independent or substantially independent of a power supply or cable terminals. The patient-securing strap may be used flexibly at all kinds of locations, for example, in an ambulance, in an examination device, in a ward, or at an accident site.

In another embodiment, the data transmission link is cable-based. The patient-securing strap includes a releasable electric connector by which at least the data transmission link is connected. The cable-based data transmission link includes a cable link, which may reduce susceptibility to interference signals. The cable link may be used for supplying the patient-securing strap with electric power for operating the transmitter or electric sensors. Insusceptibility to interference signals and dispensing with a possibly exhaustible power supply are particularly significant in view of the need to insure maximum operational reliability in medical applications.

In one embodiment, a patient-securing strap is mechanically secured on one side of a patient-supporting device. The patient-securing strap provides adequate mechanical strength for securing a patient in position.

A patient-supporting device joined to the patient-securing strap is a versatile functional unit. The patient-supporting device with the patient-securing strap may be used for transporting and possibly examining the patient in imaging examination devices, for example X-ray or CT or MR or ultrasound devices, while simultaneously measuring vital functions. The patient-supporting device may be used in acute situations, for example, in the case of accidents or in ambulances or intensive-care wards.

### BRIEF DESCRIPTION OF THE DRAWINGS

FIG. 1 shows one embodiment of a patient-securing strap,

FIG. 2 shows one embodiment of electric components of a patient-securing strap,

FIG. 3 is a different view of electric components, and

FIG. 4 shows one embodiment of a patient-supporting device having a patient-securing strap.

### DETAILED DESCRIPTION

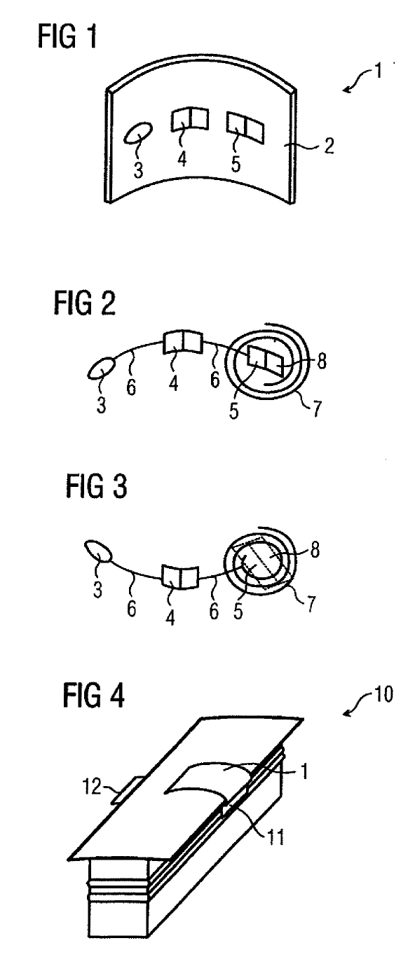

FIG. 1 shows a patient-securing strap 1. The patient-securing strap 1 may include electric sensors 3, 4, 5. In FIG. 1, the measuring areas of electric sensors 3, 4, 5, are shown. The side of the patient-securing strap 1 facing the patient is shown. The sensors 3, 4, 5 may be distributed as shown in FIG. 1 or in any positions and spatial distributions depending on the type of sensor.

The strap 1 may include a belt band 2 made of flexible plastic. For example, the belt band 2 may be made of neoprene, which may be sufficiently thick for accommodating electric components and, have pleasant tactile properties, for example, a certain suppleness and softness. Although any thickness and width may be used, it is preferable that the thickness is approximately 1 cm to 2 cm and the width is approximately 400 mm. A thickness of approximately 1 cm to 2 cm may provide sufficient room for electronic components and sufficient tensile strength. The strap 1 may be slightly bent or curved to account for the rigidity accompanying the thickness.

The sensor areas serve to register physiological measurements on the patient or as close to the patient as possible. Vital functions, such as pulse, heart rate, blood-oxygen saturation, or blood pressure, may be registered as physiological measurements. Corresponding electric sensors 3, 4, 5 may be based on temperature sensors, ECG sensors, and pressure or acoustic sensors. Blood-oxygen saturation may, through optical measuring in the infrared-wavelength range, be measured non-invasively. Sensors for the cited and further measurements are known.

FIG. 2 shows the patient-securing strap's electric components. The sensor surfaces of the electric sensors 3, 4, 5 are shown. An electric conductor 6 links the sensors 3, 4, 5, such that sensor signals may be transmitted over the conductor 6. A transponder 8 may externally transmit information in response to receiving an external signal. The transponder 8 may externally transmit sensor data via the antenna 7, which acts as both a receiving and a transmitting antenna.

The conductor 6 may be made of an X-ray-transparent material, such that the patient-securing strap 1 will not produce any image artifacts during X-ray examinations. The conductor 6 may be flexible (as compared to the belt band 2), so as to accommodate bending or pulling strains. The conductor 6 may be worked into the strap material in meandering or snaking fashion. The meanders provide flexibility in both the bending and the pulling direction.

In one embodiment, the conductor 6 may be made of polyacethylene suitably doped for providing the conductivity, Suitable doping may use iodine and be performed by, for example, vapor-depositing iodine. Because polyacethylene tends to oxidize from atmospheric oxygen, an oxidation-inhibiting sheath must be provided. Alternatively, the conductor 6 may be made of polyanilin, polypyrol, or polythiophen, which are conductive plastics that exhibit properties comparable to polyacethylene. Alongside X-ray transparency, the plastic-based conductors have the advantages typical of such systems, for example cheap and straightforward production by printing conductors using dissolved, liquid plastic material.

FIG. 3 is a different view of the electric structure, described in FIG. 2, of the patient-securing strap 1. In FIG. 3, the electric sensor 5 and transponder 8 are located beneath and concealed by the antenna 7.

FIG. 4 shows a patient-supporting device 10. A patient-securing strap 1 has been permanently attached to the patient-supporting device 10. The patient-supporting device 1 may include a height-adjustable foot mechanism. The patient-supporting device 1 may be an X-ray table or OP table. Other applications and table variants suitable for being used with the patient-securing strap 1 are conceivable.

The patient-securing strap 1 may be secured to a side of the patient-supporting device 10 by a securing device 11. The securing device 11 may be sufficiently stable to withstand the mechanical strain due to a patient's being secured in position on the patient-supporting device 10. Securing in position may be realized by, for example, a tensioning or clamping device. Other securing mechanisms are conceivable, such as screw connections or hook and latch fasteners (e.g., Velcro® fasteners). To secure a patient in position, the patient-securing strap may be placed over the patient and secured in position on the opposite side in the fastener 12. The fastener 12 may include clamping or tensioning devices, hook and latch fasteners (e.g., Velcro® fasteners), and other suitable fasteners. The patient-securing strap 1 may be used to secure a patient in an examination position on the patient-supporting device 10.

The patient-securing strap 1 may be released from the patient-supporting device on the side containing the fastener 12. Securing on the side containing the securing device 11 may be implemented durably. The electric components of the patient-securing strap 1 may be connected via a cable link in the region of the securing device 11, for example, for transmitting data or supplying power.

Summarizing, the present embodiments relate to a patient-securing strap and to a patient-supporting device having a patient-securing strap. In an exemplary embodiment the patient-securing strap (1) is made substantially of plastic and has an electric conductor (6) made of substantially X-ray-transparent plastic, an electric sensor (3, 4, 5) that is connected to the conductor (6) and made of substantially X-ray-transparent material, and a transmitter that is connected to the conductor (6) and embodied for transmitting sensor signals fed out by the sensor (3, 4, 5) onto the conductor (6) onto a data transmission link. In another exemplary embodiment a patient-supporting device (10) has a patient-securing strap (1) of such kind. Being made for the most part of an X-ray-transparent material, the patient-securing strap will not give rise to any disruptive image artifacts during an X-ray examination. It can consequently readily be used for an X-ray or CT examination performed on the patient and will at the same time allow measurements to be registered by means of the electric sensor. It will hence in other words be rendered possible to conduct an X-ray examination and simultaneously register measurements.

Various embodiments described herein can be used alone or in combination with one another. The forgoing detailed description has described only a few of the many possible implementations of the present invention. For this reason, this detailed description is intended by way of illustration, and not by way of limitation. It is only the following claims, including all equivalents that are intended to define the scope of this invention.

1. A patient-securing strap made substantially of plastic, the patient-securing strap comprising:
an electric conductor made of substantially X-ray-transparent plastic,
an electric sensor connected to the electric conductor and made of substantially X-ray-transparent material, and
a transmitter connected to the electric conductor and operable to transmit sensor signals from the sensor to the conductor and a data transmission link.

2. The patient-securing strap as claimed in claim 1, characterized in that the patient-securing strap is made substantially of neoprene.

3. The patient-securing strap as claimed in claim 1, characterized in that the conductor is made substantially of doped polyacethylene.

4. The patient-securing strap as claimed in claim 3, characterized in that the conductor includes oxidation-inhibiting sheath.

5. The patient-securing strap as claimed in claim 3, characterized in that the polyacethylene is doped with iodine.

6. The patient-securing strap as claimed in claim 1, characterized in that the conductor is made substantially of polyanilin, polypyrol, or polythiophen.

7. The patient-securing strap as claimed in claim 1, characterized in that the electric sensor operable to measure a pulse, a heart rate, a body temperature, an amount of body perspiration, a blood-oxygen saturation, a percentage of body fat, or a blood pressure.

8. The patient-securing strap as claimed in claim 1, characterized in that it has a plurality of electric sensors.

9. The patient-securing strap as claimed in claim 1, characterized in that the data transmission link is cableless and the transmitter is a transponder.

10. The patient-securing strap as claimed in claim 1, characterized in that the data transmission link includes a cable and the patient-securing strap includes a releasable electric connector that is operable to at least the data transmission link.

11. A patient-supporting system comprising:
a patient-supporting device for supporting a patient; and
a patient-securing strap made substantially of plastic and including:
an electric conductor made of substantially X-ray-transparent plastic,
an electric sensor that is connected to the conductor and made of substantially X-ray-transparent material, and
a transmitter that is connected to the conductor and operable for transmitting sensor signals from the sensor to the conductor and/or a data transmission link.

12. The patient-supporting device as claimed in claim 11, characterized in that the patient-securing strap is made substantially of neoprene.

13. The patient-supporting device as claimed in claim 11, characterized in that the patient-securing strap is mechanically secured on a side of the patient-supporting device, such that a patient is secured in an examination position.

14. The patient-supporting device as claimed in claim 13, characterized in that the patient-securing strap is electrically connected to the patient-supporting device, such that a cable-based electric connection of the data transmission link and/or a power supply may be established.

15. The patient-supporting device as claimed in claim 11, characterized in that the conductor is made substantially of doped polyacethylene.

16. The patient-supporting device as claimed in claim 11, characterized in that the patient-securing strap includes a plurality of electric sensors.

17. The patient-securing strap as claimed in claim 4, characterized in that the polyacethylene is doped with iodine.

18. The patient-securing strap as claimed in claim 2, characterized in that the conductor is made substantially of polyanilin, polypyrol, or polythiophen.

19. A patient-supporting device comprising:
a patient-securing strap made substantially of plastic and including:
an electric conductor made of substantially X-ray-transparent plastic,
an electric sensor that is connected to the conductor and made of substantially X-ray-transparent material, and
a transmitter that is connected to the conductor and operable for transmitting sensor signals from the sensor to the conductor and/or a data transmission link.
